SEQUENCE LISTING (54 sequences)

SEQ ID NO: 1 - protein, 62 aa, artificial sequence
  Polypeptide from flagellin of Salmonella enterica sub.sp enterica serovar Edimburgh Acc. No. ADU54190
XXXXXXXXXXXXXXXXXXXXXXXXXXXXXXXXXXXXXXXXXXXXXXXXXXXXXXXXXXXXXX

SEQ ID NO: 2 - protein, 62 aa, Artificial sequence
  polypeptide from flagellin of Salmonella enterica subsp. enterica serovar Edimburgh Acc. No.  ADU54190
LTIQVGANDGETIDIDLKQINSQTLSSSGSSGSSGSSIDAALAQVDALRSDLGAVQNRFNSA

SEQ ID NO: 3 - protein, 25 aa, Artificial Sequence
  X is Val, Leu, Ile or Asn
XXXXXXXXXXXXXXXXXXXXXXXXX

SEQ ID NO: 4 - protein, 25 aa, Artificial sequence
  Polypeptide from flagellin of Salmonella enterica sub.sp enterica serovar  Edimburgh Acc. No.  ADU54190
IRIMIIVIDIERIDIDVAMIVMQRI

SEQ ID NO: 5 - protein, 25 aa, Artificial sequence
  Polypeptide from flagellin of Salmonella enterica sub.sp enterica serovar  Edimburgh Acc. No.  ADU54190
IRIMIIVIDIGRIDIDVAMIVMQRI

SEQ ID NO: 6 - protein, 25 aa, Artificial sequence
  Polypeptide from flagellin of Salmonella enterica sub.sp enterica serovar  Edimburgh Acc. No.  ADU54190
IRIMINVIDIERIDIDVAMIVMQRI

SEQ ID NO: 7 - protein, 25 aa, Artificial sequence
  Polypeptide from flagellin of Salmonella enterica sub.sp enterica serovar  Edimburgh Acc. No.  ADU54190
IRIMIVVIDIERIDIDVAMIVMQRI

SEQ ID NO: 8 - protein, 25 aa, Artificial sequence
  Polypeptide from flagellin of Salmonella enterica sub.sp enterica serovar  Edimburgh Acc. No.  ADU54190
IRIMINVIDIGRIDIDVAMIVMQRI

SEQ ID NO: 9 - protein, 25 aa, Artificial sequence
  Polypeptide from flagellin of Salmonella enterica sub.sp enterica serovar Edimburgh Acc. No.  ADU54190
IRIMIVVIDIGRIDIDVAMIVMQRI

SEQ ID NO: 10 - protein, 25 aa, Artificial sequence
  Polypeptide from flagellin of Salmonella enterica sub.sp enterica serovar  Edimburgh Acc. No.  ADU54190
IRIMIIVIDIERIDIDVAMIVQQRI

SEQ ID NO: 11 - protein, 25 aa, Artificial sequence
  Polypeptide from flagellin of Salmonella enterica sub.sp enterica serovar  Edimburgh Acc. No.  ADU54190
IRIMIIVIDIERIDIDNAMIVMQRI

SEQ ID NO: 12 - protein, 25 aa, Artificial sequence
  Polypeptide from flagellin of Salmonella enterica sub.sp enterica serovar  Edimburgh Acc. No.  ADU54190
IRIMIIVIDIGRIDIDVAMIVQQRI

SEQ ID NO: 13 - protein, 25 aa, Artificial sequence
  Polypeptide from flagellin of Salmonella enterica sub.sp enterica serovar  Edimburgh Acc. No.  ADU54190
IRIMINVIDIERIDIDVAMIVQQRI

SEQ ID NO: 14 - protein, 25 aa, Artificial sequence
  Polypeptide from flagellin of Salmonella enterica sub.sp enterica serovar Edimburgh Acc. No.  ADU54190
IRIMIVVIDIERIDIDVAMIVQQRI

SEQ ID NO: 15 - protein, 25 aa, Artificial sequence
  Polypeptide from flagellin of Salmonella enterica sub.sp enterica serovar  Edimburgh Acc. No.  ADU54190
IRIMIIVIDIGRIDIDNAMIVMQRI

SEQ ID NO: 16 - protein, 25 aa, Artificial sequence
  Polypeptide from flagellin of Salmonella enterica sub.sp enterica serovar  Edimburgh Acc. No.  ADU54190
IRIMINVIDIERIDIDNAMIVMQRI

SEQ ID NO: 17 - protein, 25 aa, Artificial sequence
  Polypeptide from flagellin of Salmonella enterica sub.sp enterica serovar  Edimburgh Acc. No.  ADU54190
IRIMINVIDIGRIDIDVAMIVQQRI

SEQ ID NO: 18 - protein, 25 aa, Artificial sequence
  Polypeptide from flagellin of Salmonella enterica sub.sp enterica serovar  Edimburgh Acc. No.  ADU54190
IRIMIVVIDIERIDIDNAMIVMQRI

SEQ ID NO: 19 - protein, 25 aa, Artificial sequence
  Polypeptide from flagellin of Salmonella enterica sub.sp enterica serovar  Edimburgh Acc. No.  ADU54190
IRIMIVVIDIGRIDIDVAMIVQQRI

SEQ ID NO: 20 - protein, 25 aa, Artificial sequence
  Polypeptide from flagellin of Salmonella enterica sub.sp enterica serovar  Edimburgh Acc. No.  ADU54190
IRIMIIVIDIETIDIDVAMIVMQRI

SEQ ID NO: 21 - protein, 25 aa, Artificial sequence
  Polypeptide from flagellin of Salmonella enterica sub.sp enterica serovar Edimburgh Acc. No.  ADU54190
LTIQVGANDGETIDIDLKQINSQTL

SEQ ID NO: 22 - protein, 25 aa, Artificial sequence
  Polypeptide from flagellin of Salmonella enterica sub.sp enterica serovar  Edimburgh Acc. No.  ADU54190
IRIMIVVIDIGRIDIDNAMIVMQRI

SEQ ID NO: 23 - protein, 25 aa, Artificial sequence
  Polypeptide from flagellin of Salmonella enterica sub.sp enterica serovar  Edimburgh Acc. No.  ADU54190
IDIMIIVIDIGTIDIDVAMIVMQRI

SEQ ID NO: 24 - protein, 25 aa, Artificial sequence
  Polypeptide from flagellin of Salmonella enterica sub.sp enterica serovar  Edimburgh Acc. No.  ADU54190
NRIMIIVIDIGRIDIDVAMIVMQRI

SEQ ID NO: 25 - protein, 25 aa, Artificial sequence
  Polypeptide from flagellin of Salmonella enterica sub.sp enterica serovar  Edimburgh Acc. No.  ADU54190
IDIMINVIDIETIDIDVAMIVMQRI

SEQ ID NO: 26 - protein, 25 aa, Artificial sequence
  Polypeptide from flagellin of Salmonella enterica sub.sp enterica serovar  Edimburgh Acc. No.  ADU54190
NRIMINVIDIERIDIDVAMIVMQRI

SEQ ID NO: 27 - protein, 25 aa, Artificial sequence
  Polypeptide from flagellin of Salmonella enterica sub.sp enterica serovar  Edimburgh Acc. No.  ADU54190
IRIMIIVIDIERIDIDNAMIVQQRI

SEQ ID NO: 28 - protein, 25 aa, Artificial sequence
  Polypeptide from flagellin of Salmonella enterica sub.sp enterica serovar  Edimburgh Acc. No.  ADU54190
IDAALAQVDALRSDLGAVQNRFNSA

SEQ ID NO: 29 - protein, 25 aa, Artificial sequence
  Polypeptide from flagellin of Salmonella enterica sub.sp enterica serovar  Edimburgh Acc. No.  ADU54190
IRIMIIVIDIERIDIDVAMINMQRI

SEQ ID NO: 30 - protein, 25 aa, Artificial sequence
  Polypeptide from flagellin of Salmonella enterica sub.sp enterica serovar  Edimburgh Acc. No.  ADU54190
NRIMIVVIDIERIDIDVAMIVMQRI

SEQ ID NO: 31 - protein, 25 aa, Artificial sequence
  Polypeptide from flagellin of Salmonella enterica sub.sp enterica serovar  Edimburgh Acc. No.  ADU54190
IRIMIVVIDIETIDIDVAMIVMQRI

SEQ ID NO: 32 - protein, 25 aa, Artificial sequence
  Polypeptide from flagellin of Salmonella enterica sub.sp enterica serovar  Edimburgh Acc. No.  ADU54190
IRIMINVIDIGTIDIDVAMIVMQRI

SEQ ID NO: 33 - protein, 25 aa, Artificial sequence
  Polypeptide from flagellin of Salmonella enterica sub.sp enterica serovar  Edimburgh Acc. No.  ADU54190
NRIMINVIDIGRIDIDVAMIVMQRI

SEQ ID NO: 34 - protein, 25 aa, Artificial sequence
  Polypeptide from flagellin of Salmonella enterica sub.sp enterica serovar  Edimburgh Acc. No.  ADU54190
IRIMINVIDIERIDIDNAMIVQQRI

SEQ ID NO: 35 - protein, 25 aa, Artificial sequence
  Polypeptide from flagellin of Salmonella enterica sub.sp enterica serovar  Edimburgh Acc. No.  ADU54190
IRIMIIVIDIGRIDIDNAMIVQQRI

SEQ ID NO: 36 - protein, 25 aa, Artificial sequence
  Polypeptide from flagellin of Salmonella enterica sub.sp enterica serovar  Edimburgh Acc. No.  ADU54190
IRIMIIVIDIGRIDIDVAMINMQRI

SEQ ID NO: 37 - protein, 25 aa, Artificial sequence
  Polypeptide from flagellin of Salmonella enterica sub.sp enterica serovar  Edimburgh Acc. No.  ADU54190
NRIMIVVIDIGRIDIDVAMIVMQRI

SEQ ID NO: 38 - protein, 25 aa, Artificial sequence
  Polypeptide from flagellin of Salmonella enterica sub.sp enterica serovar  Edimburgh Acc. No.  ADU54190
IRIMIVVIDIGTIDIDVAMIVMQRI

SEQ ID NO: 39 - protein, 25 aa, Artificial sequence
  Polypeptide from flagellin of Salmonella enterica sub.sp enterica serovar Edimburgh Acc. No.  ADU54190
IRIMIVVIDIERIDIDNAMIVQQRI

SEQ ID NO: 40 - protein, 25 aa, Artificial sequence
  Polypeptide from flagellin of Salmonella enterica sub.sp enterica serovar  Edimburgh Acc. No.  ADU54190
IRIMINVIDIERIDIDVAMINMQRI

SEQ ID NO: 41 - protein, 25 aa, Artificial sequence
  Polypeptide from flagellin of Salmonella enterica sub.sp enterica serovar  Edimburgh Acc. No.  ADU54190
IRIMINVIDIGRIDIDNAMIVQQRI

SEQ ID NO: 42 - protein, 25 aa, Artificial sequence
  Polypeptide from flagellin of Salmonella enterica sub.sp enterica serovar Edimburgh Acc. No.  ADU54190
NRIMIIVIDIERIDIDVAMIVQQRI

SEQ ID NO: 43 - protein, 25 aa, Artificial sequence
  Polypeptide from flagellin of Salmonella enterica sub.sp enterica serovar  Edimburgh Acc. No.  ADU54190
IRIMIINIDIERIDIDVAMIVMQRI

SEQ ID NO: 44 - protein, 25 aa, Artificial sequence
  Polypeptide from flagellin of Salmonella enterica sub.sp enterica serovar  Edimburgh Acc. No.  ADU54190
IRIMIIVIDIETIDIDVAMIVQQRI

SEQ ID NO: 45 - protein, 25 aa, Artificial sequence
  Polypeptide from flagellin of Salmonella enterica sub.sp enterica serovar  Edimburgh Acc. No.  ADU54190
IRIMIVVIDIERIDIDVAMINMQRI

SEQ ID NO: 46 - protein, 25 aa, Artificial sequence
  Polypeptide from flagellin of Salmonella enterica sub.sp enterica serovar  Edimburgh Acc. No.  ADU54190
VRIMIIVIDIERIDIDVAMIVMQRI

SEQ ID NO: 47 - protein, 25 aa, Artificial sequence
  Polypeptide from flagellin of Salmonella enterica sub.sp enterica serovar  Edimburgh Acc. No.  ADU54190
LRIMIVVIDIGRIDIDNAMIVQQRI

SEQ ID NO: 48 - protein, 25 aa, Artificial sequence
  Polypeptide from flagellin of Salmonella enterica sub.sp enterica serovar  Edimburgh Acc. No.  ADU54190
LRIMINVIDIGRIDIDVAMINMQRI

SEQ ID NO: 49 - protein, 25 aa, Artificial sequence
  Polypeptide from flagellin of Salmonella enterica sub.sp enterica serovar  Edimburgh Acc. No.  ADU54190
LRIMIIVIDIETIDIDNAMIVMQRI

SEQ ID NO: 50 - protein, 25 aa, Artificial sequence
  Polypeptide from flagellin of Salmonella enterica sub.sp enterica serovar  Edimburgh Acc. No.  ADU54190
ITIMIIVIDIERIDIDVAMIVMQRI

SEQ ID NO: 51 - protein, 25 aa, Artificial sequence
  Polypeptide from flagellin of Salmonella enterica sub.sp enterica serovar  Edimburgh Acc. No.  ADU54190
NRIMINVIDIERIDIDVAMIVQQRI

SEQ ID NO: 52 - protein, 25 aa, Artificial sequence
  Polypeptide from flagellin of Salmonella enterica sub.sp enterica serovar  Edimburgh Acc. No.  ADU54190
NRIMIIVIDIERIDIDNAMIVMQRI

SEQ ID NO: 53 - protein, 25 aa, Artificial sequence
  Polypeptide from flagellin of Salmonella enterica sub.sp enterica serovar  Edimburgh Acc. No.  ADU54190
IRIMIINIDIGRIDIDVAMIVMQRI

SEQ ID NO: 54 - protein, 12 aa, artificial sequence
  peptide linker
SSSGSSGSSGSS

(19) United States
(12) Patent Application Publication
(10) Pub. No.: US 2017/0051021 A1
(43) Pub. Date: Feb. 23, 2017

(54) PEPTIDE AGONISTS OF TOLL-LIKE RECEPTOR 5 LIGAND AND METHOD OF USE

(71) Applicants: CENTRO DI RIFERIMENTO ONCOLOGICO, Aviano (IT); VINCA INSTITUTE OF NUCLEAR SCIENCES, Belgrade (RS)

(72) Inventors: Alfonso COLOMBATTI, Aviano (IT); Roberto DOLIANA, Aviano (IT); Veljko VELJKOVIC, Belgrade (RS); Nevena VELJKOVIC, Belgrade (RS); Sanja GLISIC, Belgrade (RS); Vladimir PEROVIC, Belgrade (RS)

(73) Assignees: Vinca Institute of Nuclear Sciences, Vinca, Belgrade (RS); Centro Di Riferimento Oncologico, Aviano, Pordenone (IT)

(21) Appl. No.: 15/307,390

(22) PCT Filed: Apr. 29, 2015
(86) PCT No.: PCT/EP2015/059424

§ 371 Date: Oct. 28, 2016

(30) Foreign Application Priority Data
Apr. 30, 2014 (IT) PN2014A000022

(51) Int. Cl. C07K 14/255; A61K 45/06; G01N 33/68; A61K 39/39; A61K 47/48; A61K 38/16; C07K 16/12

(52) U.S. Cl. CPC C07K 14/255; A61K 38/164; A61K 45/06; C07K 16/1235; A61K 39/39; A61K 47/48507; G01N 33/6872; A61K 2039/55516

(57) ABSTRACT

The present invention refers to peptides functionally related to Toll-like receptor 5 (TLR-5) ligand, having a sequence of amino acids ordered by means of the protein informational analysis techniques using the informational spectrum method with reference to the informational properties of flagellin. The sequence has in the informational spectrum the frequency of virtually the same value as the frequency characteristic determining the flagellin/TLR-5 interaction.

### FIELD OF THE INVENTION

The present invention relates to peptide agonists of the Toll-like receptor 5 (TLR-5) ligand and their use in the treatment of apoptosis-mediated tissue damages in mammal. In particular, the invention refers to polypeptides selected from the protein sequence of bacterial flagellin, and their use as radioprotectant and radiomitigator in the treatment of cancer by radiation or as immune response modulator.

### BACKGROUND OF THE INVENTION

Ionising radiation can be used to critically damage and kill rapidly proliferating cancer cells. This principle is utilized during targeted radiotherapy of the diseased site. During cancer therapy, normal cells may inadvertently be exposed to radiation, leading to severe side effects. Normal cells may also be exposed to ionising radiation by other means such as occupational, environmental, or even nuclear accident or terrorist attack so the identification of agents to combat irradiation effects are imperative.

The toxicity of ionizing radiation (IR) in mammals is largely due to damage to the most radiosensitive tissues, the gastrointestinal (GI) tract and the hematopoietic (HP) system. A number of approaches and compounds have been developed to protect or restore HP, including immune, function following IR exposure. In contrast, GI radiation syndrome remains poorly treatable resulting in significant mortality and reduced quality of life for survivors.

In preliminary studies it was demonstrated that the bacterial flagellin protein, acting as an NF-kB activating agonist of Toll-like receptor 5 (TLR-5), is a powerful radioprotectant and radiomitigator capable of improving survival when given either before or after lethal IR, respectively (Burdelya L G, Krivokrysenko V I, Tallant T C, Strom E, Gleiberman A S, Gupta D, Kumasov O V, Fort F L, Osterman A L, Didonato J A, Feinstein E, Gudkov A V. Science. 2008; 320(5873):226-230). Notably, the anti-radiation effects of flagellin are associated with protection of the GI tract as well as the HP system (Jones R M, Sloane V M, Wu H, Luo L, Kumar A, Kumar M V, Gewirtz A T, Neish A S. Gut. 2011; 60(5):648-570).

Although there already are a number of candidate agents to protect, at least partially, against acute radiation damage, no one of them are licensed in EU a countermeasure for the acute radiation syndrome (ARS). In US, FDA approved CBLB502 as an orphan drug against ARS. This pharmacologically optimized TLR-5 agonist with reduced immunogenicity was generated by deleting portions of flagellin that are non-essential for TLR-5 activation. CBLB502 is effective and non-toxic in mice and non-human primates and is being developed into a stable, self-administrable radiation antidote projected for biodefense applications. The main properties of CBLB502 are the following: (i) acts through multiple mechanisms mediated by activation of pro-survival NF-kB signaling pathway, (ii) selectively protects normal tissues (but not malignant tumors) from radiation, (iii) increases survival of stem cells and early progenitors of hematopoietic system and stimulates regeneration of different HP lineages, and (iv) reduces radiation damage to and stimulate regeneration of crypts, villi and lamina propria of GI tract.

TLR-5 activation by flagellin mediates innate immune response to elicit potent antitumor activity in cancer cells (Galli R, Starace D, Bush R, Angelini D F, Paone A, De Cesaris P, Filippini A, Sette C, Battistini L, Ziparo E, Riccioli A. J Immunol. 2010; 184(12):6658-6669; Cai Z, Sanchez A, Shi Z, Zhang T, Liu M, Zhang D. Cancer Res. 2011; 71(7):2466-2475), thereby flagellin also represents a tool to enhance the efficacy of conventional therapies by stimulating anticancer immune responses.

TLR-5 mediates the mammalian innate immune response to flagellin, and provides defense against infections caused by many different species of flagellated bacteria (Smith K D, Ozinsky A. Curr Top Microbiol Immunol. 2002; 270:93-108).

TLR-5 agonists can serve targets suitable for the development of various immune regulators, particularly vaccine adjuvants (Mizel S B, Bates J T. J Immunol. 2010; 185(10):5677-5682). As used herein, the term “vaccine adjuvants” refers to substances which can enhance, prolong or accelerate Ag-specific immune responses induced by vaccine antigens, when they are co-administered with vaccines.

In particular, as shown in FIG. 12, it is known that flagellin has conserved domains (ND1 and CD1) at the N terminus and C terminus and a middle hypervariable domain (D2) (Samatey, et al 2001, Eaves-Pyles T, et al 2001). These regions have been demonstrated to provide a potential activity. Therefore, as stated above, CBLB502 polypeptide has been synthetized in order to obtain the unique drug approved by FDA against ARS. However, as shown in FIG. 12, CBLB502 is a synthetic polypeptide consisting of the first 176 amino acids of flagellin at the N terminus linked to the last 49 amino acid at its C terminus through a flexible linker. It is evident that said construct is quite large. Accordingly, its production is particularly laborious and, consequently, may easily result in errors and wasting time.

### SUMMARY OF THE INVENTION

The scope of the present invention is to provide a new and effective composition comprising a pharmaceutically effective amount of flagellin agonist (i) for protection of a mammal from one or more treatments or conditions that trigger apoptosis and (ii) for stimulation of innate immune response in a mammal against cancer and infectious agents. Said composition should be produced in a fast, simple and reliable manner, avoiding laborious production steps.

The flagellin agonist having an enhanced activity of stimulating TLR-5 may comprise any sequence derived from the general formula SEQ ID NO: 1 or SEQ ID NO 3 or a fragment, variant, analog, homolog, derivative of SEQ ID NO: 1 or SEQ ID NO 3, or combination thereof, presenting in the informational spectrum as a dominant the frequency component F in the range 0.404-0.407. In particular, said agonist is an isolated polypeptide having the general formula R1-L-R2, wherein R1 is an amino terminal sequence, L is flexible peptide linker comprising 10-14 amino acids, R2 is the carboxy terminal sequence, and wherein both R1 and R2 have in their informational spectrum the dominant frequency component belonging to the frequency interval 0.404-0.407.

Preferably, R1 sequence consists in the general formula X11-X12-X13-X14-X15-X16-X17-X18-X19-X110-X111-X112-X113-X114-X115-X116-117-X118-X119-X120-X121-X122-X123-X124-X125, where X11 is Val, Leu, lie, Asn or Gly, X12 is Thr, Phe or Arg, X13 is Val, Leu, lie, Asn or Gly, X14 is Met, Ser, Gln or Cys, X15 is Val, Leu, lie, Asn or Gly, X16 is Val, Leu, lie, Asn or Gly, X17 is Val, Leu, lie, Asn, Gly or Ala, X18 is Val, Leu, lie, Asn or Gly, X19 is Asp, X110 is Val, Leu, lie, Asn or Gly, X111 is Gly, Glu or Val, X112 is Thr, Phe or Arg, X113 is Val, Leu, lie, Asn or Gly, X114 is Asp, X115 is Val, Leu, Ile, Asn or Gly, X116 is Asp, X117 is Val, Leu, lie, Asn or Gly, X118 is Ala or Lys, X119 is Gln, Met, Ser or Cys, X120 is Val, Leu, Ile, Asn or Gly, X121 is Val, Leu, Ile, Asn or Gly, X122 is Met, Ser, Gln or Cys, X123 is Met, Ser, Gln or Cys, X124 is Thr, Phe or Arg and X125 is Val, Leu, le, Asn or Gly.

R2 sequence consists in the general formula X21-X22-X23-X24-X25-X26-X27-X28-X29-X210-X211-X212-X213-X214-X215-X216-X217-X218-X219-X220-X221-X222-X223-X224-X225, where X21 is Val, Leu, Ile, Asn or Gly, X22 is Asp, X23 is Ala or Lys, X24 is Ala or Lys, X25 is Val, Leu, lie, Asn or Gly, X26 is Ala or Lys, X27 is Gln, Met, Ser or Cys, X28 is Val, Leu, Ile, Asn or Gly, X29 is Asp, X210 is Ala or Lys, X211 is Val, Leu, le, Asn or Gly, X212 is Thr, Phe or Arg, X213 is Gln, Met, Ser or Cys, X214 is Asp, X215 is Val, Leu, lie, Asn or Gly, X216 is Val, Leu, lie, Asn or Gly, X217 is Ala or Lys, X218 Val, Leu, lie, Asn or Gly, X219 is Gln, Met, Ser or Cys, X220 is Val, Leu, Ile, Asn or Gly, X221 is Thr, Phe or Arg, X222 is Thr, Phe or Arg, X223 is Val, Leu, Ile, Asn or Gly, X224 is Gln, Met, Ser or Cys and X225 is Ala or Lys. The peptide linker L consists in the amino acid sequence SSSGSSGSSGSS (SEQ ID NO 54).

More preferably, R1 and R2 sequences consist in the general formula X1-X2-X3-X4-X5-X6-X7-X8-X9-X10-X11-X12-X13-X14-X15-X16-17-X18-X19-X20-X21-X22-X23-X24-X25 (SEQ ID NO 3), wherein X1 is Val, Leu, Ile or Asn, X2 is Thr or Arg, X3 is Val, Leu, lie or Asn, X4 is Met or Gln, X5 is Val, Leu, Ile or Asn, X6 is Val, Leu, lie or Asn, X7 is Val, Leu, lie, Asn or Ala, X8 is Val, Leu, lie or Asn, X9 is Asp, X10 is Val, Leu, Ile or Asn, X11 is Gly or Glu, X12 is Thr or Arg, X13 is Val, Leu, lie or Asn, X14 is Asp, X15 is Val, Leu, lie or Asn, X16 is Asp, X17 is Val, Leu, lie or Asn, X18 is Ala, X19 is Gln or Met, X20 is Val, Leu, lie or Asn, X21 is Val, Leu, Ile or Asn, X22 is Met or Gln, X23 is Met or Gln, X24 is Thr or Arg and X25 is Val, Leu, lie or Asn. In particular, R1 and R2 sequences are selected from the group consisting in: IRIMIIVIDIERIDIDVAMIVMQRI (SEQ ID NO 4), IRIMIIVIDIGRIDIDVAMIVMQRI (SEQ ID NO 5), IRIMINVIDIERIDIDVAMIVMQRI (SEQ ID NO 6), IRIMIWIDIERIDIDVAMIVMQRI (SEQ ID NO 7), IRIMINVIDIGRIDIDVAMIVMQRI (SEQ ID NO 8), IRIMIWIDIGRIDIDVAMIVMQRI (SEQ ID NO 9), IRIMIIVIDIERIDIDVAMIVQQRI (SEQ ID NO 10), IRIMIIVIDIERIDIDNAMIVMQRI (SEQ ID NO 11), IRIMIIVIDIGRIDIDVAMIVQQRI (SEQ ID NO 12), IRIMINVIDIERIDIDVAMIVQQRI (SEQ ID NO 13), IRIMIVVIDIERIDIDVAMIVQQRI (SEQ ID NO 14),

(SEQ ID NO 15)
IRIMIIVIDIGRIDIDNAMIVMQRI,
(SEQ ID NO 16)
IRIMINVIDIERIDIDNAMIVMQRI,
(SEQ ID NO 17)
IRIMINVIDIGRIDIDVAMIVQQRI,
(SEQ ID NO 18)
IRIMIVVIDIERIDIDNAMIVMQRI
(SEQ ID NO 19)
IRIMIVVIDIGRIDIDVAMIVQQRI,
(SEQ ID NO 20)
IRIMIIVIDIETIDIDVAMIVMQRI,
(SEQ ID NO 21)
LTIQVGANDGETIDIDLKQINSQTL,
(SEQ ID NO 22)
IRIMIVVIDIGRIDIDNAMIVMQRI,
(SEQ ID NO 23)
IDIMIIVIDIGTIDIDVAMIVMQRI,
(SEQ ID NO 24)
NRIMIIVIDIGRIDIDVAMIVMQRI,
(SEQ ID NO 25)
IDIMINVIDIETIDIDVAMIVMQRI,
(SEQ ID NO 26)
NRIMINVIDIERIDIDVAMIVMQRI,
(SEQ ID NO 27)
IRIMIIVIDIERIDIDNAMIVQQRI,
(SEQ ID NO 28)
IDAALAQVDALRSDLGAVQNRFNSA,
(SEQ ID NO 29)
IRIMIIVIDIERIDIDVAMINMQRI,
(SEQ ID NO 30)
NRIMIVVIDIERIDIDVAMIVMQRI,
(SEQ ID NO 31)
IRIMIVVIDIETIDIDVAMIVMQRI,
(SEQ ID NO 32)
IRIMINVIDIGTIDIDVAMIVMQRI,
(SEQ ID NO 33)
NRIMINVIDIGRIDIDVAMIVMQRI,
(SEQ ID NO 34)
IRIMINVIDIERIDIDNAMIVQQRI,
(SEQ ID NO 35)
IRIMIIVIDIGRIDIDNAMIVQQRI,
(SEQ ID NO 36)
IRIMIIVIDIGRIDIDVAMINMQRI,
(SEQ ID NO 37)
NRIMIVVIDIGRIDIDVAMIVMQRI,
(SEQ ID NO 38)
IRIMIVVIDIGTIDIDVAMIVMQRI,
(SEQ ID NO 39)
IRIMIVVIDIERIDIDNAMIVQQRI,
(SEQ ID NO 40)
IRIMINVIDIERIDIDVAMINMQRI,
(SEQ ID NO 41)
IRIMINVIDIGRIDIDNAMIVQQRI,
(SEQ ID NO 42)
NRIMIIVIDIERIDIDVAMIVQQRI,
(SEQ ID NO 43)
IRIMIINIDIERIDIDVAMIVMQRI,
(SEQ ID NO 44)
IRIMIIVIDIETIDIDVAMIVQQRI,
(SEQ ID NO 45)
IRIMIVVIDIERIDIDVAMINMQRI,
(SEQ ID NO 46)
VRIMIIVIDIERIDIDVAMIVMQRI,
(SEQ ID NO 47)
LRIMIVVIDIGRIDIDNAMIVQQRI,
(SEQ ID NO 48)
LRIMINVIDIGRIDIDVAMINMQRI,
(SEQ ID NO 49)
LRIMIIVIDIETIDIDNAMIVMQRI,
(SEQ ID NO 50)
ITIMIIVIDIERIDIDVAMIVMQRI,
(SEQ ID NO 51)
NRIMINVIDIERIDIDVAMIVQQRI,
(SEQ ID NO 52)
NRIMIIVIDIERIDIDNAMIVMQRI,
(SEQ ID NO 53)
IRIMIINIDIGRIDIDVAMIVMQRI,

The flagellin agonist may be used to treat a patient undergoing cancer treatment, which may be chemotherapy or radiation therapy. The agent may be administered prior to, together with, or after a treatment for the cancer. The flagellin agonist may be used to treat a mammal exposed to radiation. The flagellin agonist may be used to treat a mammal from bacterial and viral infection.

This invention also relates to a method of modulating cell aging in a mammal, comprising administering to the mammal a composition comprising a pharmaceutically acceptable amount of an agent which modulates apoptosis. The agent may be flagellin agonist. The agent may be administered prior to, together with, or after a treatment for a disease suffered by the mammal.

The present invention provides flagellin agonists as adjuvants, vaccines and related methods that are useful in eliciting immune responses, particularly immune responses against tumor antigens. The tumor antigen may be from a tumor selected from the group consisting of leukemia, lymphoma, lung cancer, prostate cancer, colorectal cancer, thyroid cancer, renal cancer, adrenal cancer, liver cancer, pancreatic cancer, breast cancer and central and peripheral nervous system cancer.

Another aspect of the invention provides flagellin agonists as adjuvants vaccines and related methods for inducing the immune responses against infectious agent. The infectious agent might be bacteria or virus.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

Further features and the advantages of the peptides agonist of TLR-5 of the invention will become more evident from the following description of an embodiment given as no limiting example, with reference to the following figures:

FIG. 1 shows the accession numbers of the 99 flagellin proteins available in the NCBI protein data bases;

FIG. 2 shows the amino acid sequence of the fagellin protein of Salmonella enterica subsp. enterica serovar Edinburgh according to the GenBank database accession number ADU54190;

FIG. 3 shows the informational spectrum of TLR-5, wherein the abscissa represents frequencies and the ordinate represents the signal-to noise ratio (S/N);

FIG. 4 shows the informational spectrum of flagellin from Salmonella enterica subsp. enterica serovar Edinburgh (Acce. No. ADU54190), wherein the abscissa and the ordinate are as given in FIG. 3;

FIG. 5 shows the consensus informational spectrum of 99 flagellins presented in NCBI protein database, wherein the abscissa and the ordinate are as given in FIG. 3;

FIG. 6 shows the cross-spectrum between flagellin from Salmonella enterica (ADU54190) and Toll-like receptor 5 (TLR-5), wherein the abscissa and the ordinate are as in given FIG. 3;

FIG. 7 shows the location of domains of flagellin from Salmonella enterica (ADU54190) which are essential for interaction with TLR-5, wherein the abscissa and the ordinate are as in given in FIG. 3;

FIG. 8 shows the informational spectrum of the domain ND1 of flagellin from Salmonella enterica (ADU54190) (residues 144-168), wherein the abscissa and the ordinate are as in given in FIG. 3;

FIG. 9 shows the informational spectrum of the domain CD1 of flagellin from Salmonella enterica (ADU54190) (residues 423-447), wherein the abscissa and the ordinate are as in given FIG. 3;

FIG. 10 shows the informational spectrum of the flagellin mimetic agonist (SEQ ID NO: 2) derived from the general formula (SEQ ID NO: 1 and SEQ ID NO 3);

FIG. 11 shows the cross-spectrum between the flagellin mimetic agonist (SEQ ID NO: 2) and TLR-5;

FIG. 12 shows the comparison between CBLB502 compound and VINCRO1;

FIG. 13 shows the strategy used to generate the VINCRO1 construct;

FIG. 14 shows the expression of VINCRO1;

FIG. 15 shows the activation in CACO-2 cell line of the NF-kB expression by VINCRO1;

FIG. 16 shows the ability of VINCRO1 to protect Wistar rats from lethal irradiation.

### DETAILED DESCRIPTION OF THE INVENTION

The presented invention refers to flagellin derived peptides having a sequence obtained by informational spectrum method (ISM), expressing immunomodulatory, antiapoptotic and anti-cancer activity through activation of toll-like receptor 5 (TLR-5). The design of active peptide flagellin agonists expands the available methods for treatment of cancer and radiation disease and for inducing an immune response.

In one embodiment, the invention is directed to peptide flagellin agonists that bind to TLR-5 and activate TLR-5-mediated activity. The peptides can be used, for example, to improve radiation cancer therapy, to treat radiation disease or to effectively stimulate an immune response by administration of flagellin agonists and combination of these peptides with radiation/chemo therapy, other radio protectant drugs and antigens. The advantage of peptide flagellin agonists of the invention is that they provide the activity of flagellin with the availability of rapid and efficient method for recombinant production of peptides. In fact, since the invention is directed to a polypeptide sequence and not to a protein, the design, recombinant and amplification of the same sequence can be performed using well know standard methods.

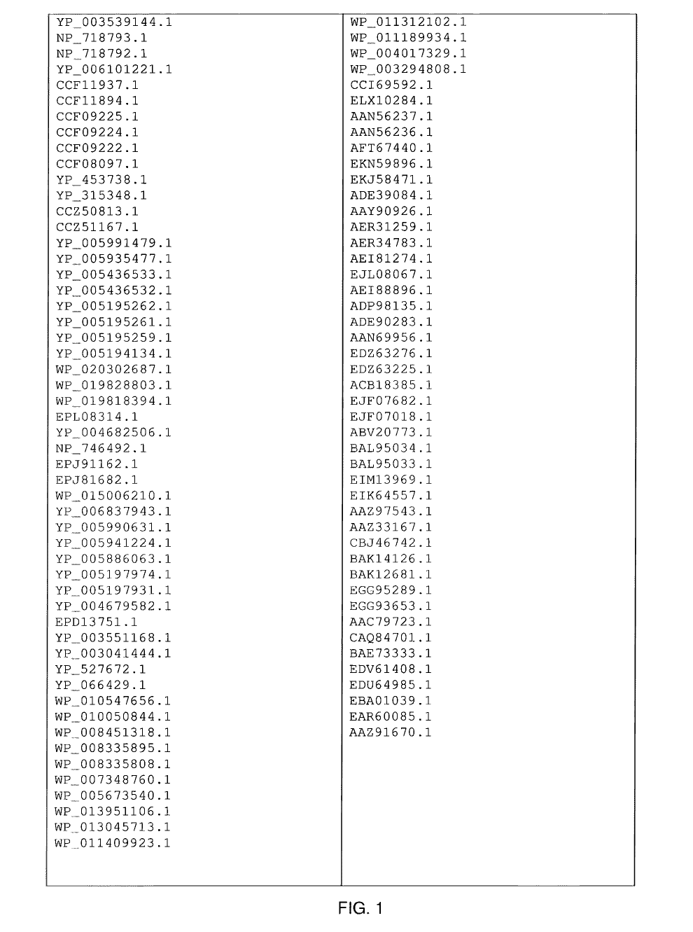

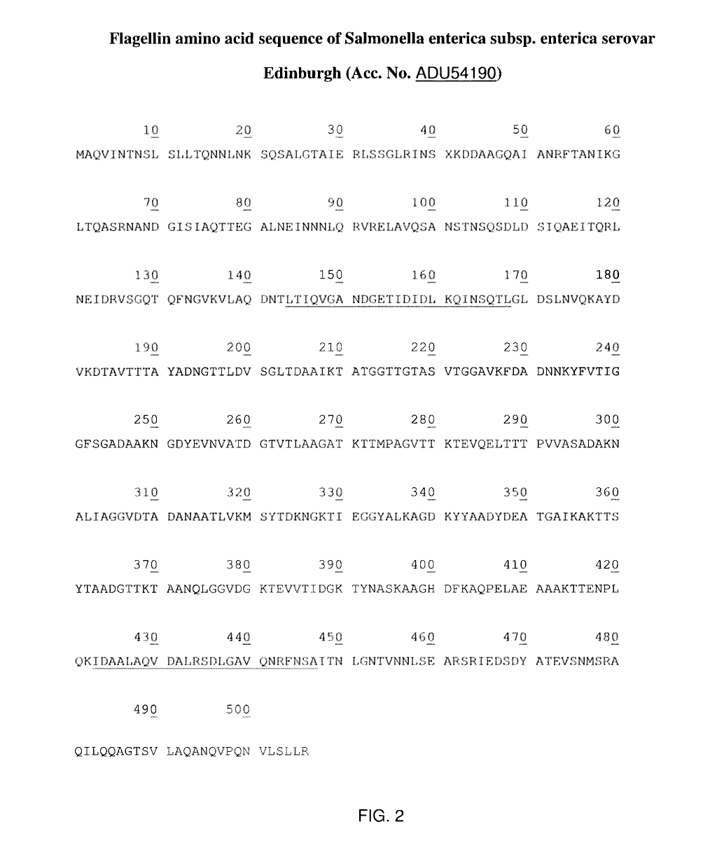

As used herein, the term “flagellin” is intended to mean a flagellin protein contained in variety of Gram-positive and Gram-negative bacterial species. The Accession Numbers of 99 flagellins publicly available in the NCBI protein database are given in FIG. 1. The flagellin sequences from these and other species are intended to be encompassed by the term flagellin as used herein. Therefore, the sequence differences between species are included within the meaning of the term.

As used herein, the term “flagellin mimetic” is intended to mean a peptide having in the informational spectrum obtained by Fourier transformation according to the informational analysis method the dominant peak on the frequency F in the range 0.404-0.407, bind TLR-5 and have substantially the same antiapoptotic, anticancer and immunomodulatory function activity of bacterial flagellin. Therefore, peptides and polypeptides mimicking the activity of bacteria flagellins are to be intended as peptides or polypeptides having the same antiapoptotic, anticancer and immunomodulatory function of a bacterial flagellin.

A flagellin agonist or modification thereof of the invention binds to TLR-5 and induces a TLR-5-mediated response. It is understood that minor modifications (such as single amino acid substitutions) can be made without destroying the TLR-5 binding activity, TLR-5-mediated response stimulating activity or immune response modulating activity, may be required in order to affect activity. Such modifications are included within the meaning of the term flagellin agonist so long as TLR-5 binding activity, TLR-5 response stimulating or immune response stimulating activities are retained. Further, various molecules can be attached to flagellin polypeptides and peptides, including for example, other polypeptides, carbohydrates, nucleic acids or lipids. Such modifications are included within the definition of the term.

Minor modifications of flagellin agonist having at least about the same TLR-5 binding activity, TLR-5 response stimulating or immune response stimulating activity as the polypeptide or peptide determined by the reference sequence (SEQ ID NO: 1), for example, conservative substitutions of naturally occurring amino acids and as well as structural alterations which incorporate non-naturally occurring amino acids, amino acid analogs and functional mimetics with the electron-ion interaction potential (EIIP) which is similar to the EIIP values (difference≦±0.005Ry) of the group of residues on the corresponding position in the reference sequence (SEQ ID NO: 1). For example, a Lysine (Lys) is considered to be a conservative substitution for the amino acid Arg. Similarly, a flagellin peptide containing mimetic structures having similar charge and spacial arrangements as reference amino acid residues would be considered a modification of the reference polypeptide or peptide so long as the peptide mimetic exhibits at least about the same activity as the reference peptide. In particular, the flagellin peptide has an identity of 50%, 60%, 70%, 80% or 90% with the SEQ ID NO 1, SEQ ID NO 2 or SEQ ID NO 3.

As used herein, the term “amino acid” is intended to mean both naturally occurring and non-naturally occurring amino acids as well as amino acid analogs and mimetics. Naturally occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like. Amino acid analogs include modified forms of naturally and non-naturally occurring amino acids. Such modifications can include, for example, substitution or replacement of chemical groups and moieties on the amino acid or by derivatization of the amino acid (e.g. o-bromo-L-phenylalanine). Amino acid mimetics include, for example, organic structures which exhibit functionally similar properties such as charge and charge spacing characteristic of the reference amino acid. For example, an organic structure which mimics Arginine (Arg or R) would have a positive charge moiety located in similar molecular space and having the same degree of mobility as the ε-amino group of the side chain of the naturally occurring Arg amino acid.

Specific examples of amino acid analogs and mimetics can be found described in, for example, Roberts and Vellaccio, The Peptides: Analysis, Synthesis, Biology, Eds. Gross and Meinhofer, Vol. 5, p. 341, Academic Press, Inc., New York, N.Y. (1983), the entire volume of which is incorporated herein by reference. Other examples include peralkylated amino acids, particularly permethylated amino acids. See, for example, Combinatorial Chemistry, Eds. Wilson and Czamik, Ch. 11, p. 235, John Wiley & Sons Inc., New York, N.Y. (1997), the entire book of which is incorporated herein by reference. Yet other examples include amino acids whose amide portion (and, therefore, the amide backbone of the resulting peptide) has been replaced, for example, by a sugar ring, steroid, benzodiazepine or carbo cycle. See, for instance, Burger's Medicinal Chemistry and Drug Discovery, Ed. Manfred E. Wolff, Ch. 15, pp. 619-620, John Wiley & Sons Inc., New York, N.Y. (1995), the entire book of which is incorporated herein by reference. Methods for synthesizing peptides, polypeptides, peptidomimetics and proteins are well known in the art (see, for example, U.S. Pat. No. 5,420,109; M. Bodanzsky, Principles of Peptide Synthesis (1st ed. & 2d rev. ed.), Springer-Verlag, New York, N.Y. (1984 & 1993), see Chapter 7; Stewart and Young, Solid Phase Peptide Synthesis, (2d ed.), Pierce Chemical Co., Rockford, III. (1984), each of which is incorporated herein by reference).

As used herein, the term “informational spectrum method” (ISM) is intended to mean a virtual spectroscopy method for analysis of protein and nucleotide sequences, which was first disclosed by Veljkovic V. et al. in IEEE Trans. Biomed. Eng. 32, 337 (1985); Cancer Biochem. Biophys. 9, 139 (1987), the content of which is herein incorporated by reference. ISM method is based on the analysis of the information encoded in primary structure of a protein or peptide, which primary structure is expressed by molecular electric oscillations propagating through polar environment. Based on the previously demonstrated strong correlation between electron-ion interaction potential (hereinafter EIIP) [Veljkovic V, Slavic I Phys. Rev. Lett. 21, 105 (1972); Veljkovic V. Phys. Lett. 45A, 41 (1973); A theoretical approach to preselection of carcinogens and chemical carcinogenesis, Gordon & Breach Sci. Pub., New York, (1980); Politzer P. and Truhlar D. G., Chemical applications of atomic and molecular electrostatic potential, Plenum Press, New York (1981); Politzer P., Toxicol, Lett. 43, 227 (1988)] it has been proposed that information expressed by electric oscillations is encoded in protein primary structure by distribution of the values of EIIP of amino acids.

According to this approach, the protein sequences are transformed into signals by assignment of numerical values to each amino acid. These values correspond to EIIP [Veljkovic V. and Slavic I., Phys. Rev. Lett., 29, 105 (1972); Veljkovic V. Phys Lett., 45A, 41 (1973)]. The signal so obtained is than decomposed in periodical function by Fourier transformation. The result is a series of frequencies and their amplitudes. The obtained frequencies correspond to the distribution of structural motifs with defined physic-chemical characteristics responsible for biological function of protein. When comparing proteins which share the same biological or biochemical function, the technique allows detection of code (amino acid)/frequency pairs which are specific for their common biological properties. The method is insensitive to the location of the motifs and, thus, does not require previous alignment of the sequence. The ISM was successfully applied to structure/function analysis of proteins, identification of protein interactors, assessment of biological effect of mutations and de novo design of peptides with desired biological function [Cosic I. and Nesis D., Eur. J. Biochem., 170, 247 (1988); Skerl V., and Pavlovic M., FEBS Lett., 239, 1411 (1988); Veljkovic V. and Metlas R., Cancer Biochem. Biophys., 10, 191 (1988); Cosic I. et al., Biochemie, 71, 333 (1989); Lalovic D. and Veljkovic V., Biosystems, 23, 311 (1989); Cosic I., Resonant recognition model of protein-protein and protem-DNA recognition, in Bioinstrumentation and Biosensor (edited by Weis D. L.), Marcel Dekker, Inc., New York (1990); Cosic I. et al., Eur. J. Biochem. 198, 113 (1991); Cosic I. and Heam M. T. W., J. Mol. Recognition., 4, 57 (1991); Veljkovic V. et al., Biochem. Biophys. Res. Commun., 189, 705 (1992); Veljkovic et al. Biochem Biophys. Res. Commun. 196, 1019 (1993); Veljkovic V. et al. J Pept Res, 62, 158 (2003); Veljkovic V. et al. Curr. Medic. Chem., 14, 441 (2007); Doliana R. et a. Matrix Biol., 27, 96 (2008); Veljkovic V. et a. Curr Protein Pept Sci. 9, 493 (2008); Veljkovic V. et al. BMC Struct Biol., 9, 21 (2009); Veljkovic V. et al. BMC Struct Biol., 9, 62 (2009); Tintori C. et al. Proteins, 78, 3396 (2010); Vasiljevic N. et al. Viral Immunol., 24:69 (2011); Glisic S. et al. Protein J, doi: 10.1007/s10930-011-9381-6 (2011); Veljkovic V. et al. Expert Opinion on Drug Discovery, doi: 0.1517/17460441.2012.638280 (2011); Glisic S. et al. Protein J. 31:129 (2012); Stojkovic L. et al. J Neuroimmunol., 245:92 (2012); Mancini M. et al. J Cell Biochem., 113:2765 (2012).

The flagellin agonist identified using the methods and compositions described herein, are potential therapeutic polypeptides that can be administered to an individual, such as a human or other mammal, in an effective amount to increase signaling through TLR-5, for example, to alter an immune response or treat a TLR-5-associated condition.

The flagellin agonists and modifications thereof, are useful for ameliorating, or reducing the severity of a pathological condition. Reduction in severity includes, for example, an arrest or decrease in clinical symptoms, physiological indicators, biochemical markers or metabolic indicators of disease caused by toxic substances or ionizing radiation (IR). Those skilled in the art will know, or will be able to determine the appropriate clinical symptoms, physiological indicators, biochemical markers or metabolic indicators to observe for a particular pathological condition. To prevent a disease means to preclude the occurrence of a disease or restoring a diseased individual to their state of health prior to disease by administration of the flagellin agonist administrated either before or after IR.

In addition to applications described herein, the flagellin agonist can be used, for example, to specifically target a diagnostic moiety to cells and tissues that express TLR-5, such as monocytes, immature dendritic cells, epithelial cells, and other cells involved in an immune response. Thus, a flagellin agonist can be labeled with a detectable moiety, such as a radiolabel, fluorochrome, ferromagnetic substance, or luminescent substance, and used to detect normal or abnormal expression of TLR-5 polypeptide in an isolated sample or in vivo diagnostic imaging procedures. For instance, the diagnostic method in vitro comprises contacting the labeled polypeptide, composition or antibodies according to the present invention with an isolated sample and detecting the eventual presence of a signal representative of the normal expression of the TLR-5 receptor.

The flagellin agonist polypeptides and peptides described herein, can be prepared using a variety of protein expression systems well known in the art, including prokaryotic and eukaryotic expression systems. Prokaryotic expression systems are advantageous due to their ease in manipulation, low complexity growth media, low cost of growth media, rapid growth rates and relatively high yields.

Well known prokaryotic expression systems include, for example, E. coli bacterial expression systems based on bacteriophage T7 RNA polymerase, the trc promoter, the araB promoter and bacillus expression. Eukaryotic expression systems are advantageous because expressed polypeptides can contain eukaryotic post-translational modifications such as O-linked glycosylation, phosphorylation and acetylation and can have improved protein folding. Well known eukaryotic expression systems include, for example, expression in yeast, such as Pichia pastoris and Pichia methanolica, expression in insect systems such as the Drosophila S2 system and baculovirus expression systems and expression in mammalian cells using adenoviral vectors and cytomegalovirus promoter-containing vectors.

The flagellin agonist polypeptides and peptides described herein can be purified using a variety of methods of protein purification well known in the art. Biochemical purification can include, for example, steps such as solubilization of the polypeptide or peptide-expressing cell, isolation of the desired subcellular fractions, chromatography, such as ion exchange, size, or affinity-based chromatographies, electrophoresis, and immunoaffinity procedures.

Other well-known methods are described in Deutscher et al., Guide to Protein Purification: Methods in Enzymology Vol. 182, (Academic Press, (1990)). An exemplary method for purifying a flagellin peptide is provided in Example 6. The methods and conditions for biochemical purification of a polypeptide of the invention can be chosen by those skilled in the art, and the purification monitored, for example, by staining SDS-PAGE gels containing protein samples, by immunodetection methods such as Western blotting and ELISA, and by functional assay of immunogenic activity of flagellin or a TLR-5 activity of TLR-5.

The flagellin agonist of the invention binds to TLR-5 and stimulates a TLR-5 activity. The ability of the flagellin agonist to bind to TLR-5 or stimulate a TLR-5 activity can be determined using methods known in the art. Methods of determining specific binding interactions of flagellin agonist with TLR-5 can be determined using well known methods in the art such as methods of trapping ligand-receptor complexes using chemical cross-linking, and competitive inhibition of reagents specific for TLR-5 such as specific flagellin peptides or modifications, antibodies or other TLR-5 specific reagents.

Methods of determining TLR-5 functional activities in response to the flagellin agonist include methods described herein, in Examples 1 through 8, as well as methods known in the art. A variety of methods well known in the art can be used for determining transcription factor activities. For example, fos, jun, and NF-KB activation in response to TLR-5 binding to a flagellin molecule can be detected by electrophoretic mobility shift assays well known in the art that detect NF-KB binding to specific polynucleic acid sequences, and promoter-reporter nucleic acid constructs such that, for example, β-lactamase, luciferase, green fluorescent protein or β-galactosidase will be expressed in response to contacting a TLR-5 with a flagellin agonist. For example, a luciferase reporter plasmid in which luciferase protein expression is driven by one or more NF-KB binding sites can be transfected into a cell, as described in Example 7. Activation of NF-KB results in activation of luciferase reporter expression, resulting in production of luciferase enzyme able to catalyze the generation of a molecule that can be detected by colorimetric, fluorescence, chemiluminescence or radiometric assay.

An amount or activity of a polypeptide, including a cytokine such as TNFα, IL-1 or IL-6, can be a read-out for activation of a TLR-5 in response to binding the flagellin agonist. A variety of methods well known in the art can be used to measure cytokine amounts, such as, for example, flow cytometry methods, immunoassays such as ELISA and RIA, and cytokine RNA protection assays. Commercially available cytokine assay kits, such as ELISA assay formats, can be conveniently used to determine the amount of a variety of cytokines in a sample. Those skilled in the art will determine the particular cytokines to be measured when assessing an immune response in a cell or animal.

For treating or reducing the severity of a pathological condition caused by IR, a flagellin agonist can be formulated and administered in a manner and in an amount appropriate for the condition to be treated; the weight, gender, age and health of the individual; the biochemical nature, bioactivity, bioavailability and side effects of the particular compound; and in a manner compatible with concurrent treatment regimens. An appropriate amount and formulation for a particular therapeutic application in humans can be extrapolated based on the activity of the compound in recognized animal models of the particular disorder.

The flagellin agonist can be administered to an individual using a variety of methods known in the art including, for example, intravenously, intramuscularly, subcutaneously, intraorbitally, intracapsularly, intraperitoneally, intracistemally, intra-articularly, intracerebrally, orally, intravaginally, rectally, topically, intranasally, or transdermally. The flagellin agonist can be administered to a subject as a pharmaceutical composition comprising the compound and a pharmaceutically acceptable carrier at any dosage including, but not limited to, about 1 μg/kg, 25 μg/kg, 50 μg/kg, 75 μg/kg, 100 μg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg or 1 mg/kg.

The choice of pharmaceutically acceptable carrier depends on the route of administration of the compound and on its particular physical and chemical characteristics. Pharmaceutically acceptable carriers are well known in the art and include sterile aqueous solvents such as physiologically buffered saline, and other solvents or vehicles such as glycols, glycerol, oils such as olive oil and injectable organic esters. A pharmaceutically acceptable carrier can further contain physiologically acceptable compounds that stabilize the compound, increase its solubility, or increase its absorption. Such physiologically acceptable compounds include carbohydrates such as glucose, sucrose or dextrans; antioxidants, such as ascorbic acid or glutathione; chelating agents; and low molecular weight proteins. In general, antioxidants should be selected among molecules having EIIP values between 0.00 and 0.10 Ry and AQVN values<2.40 or compounds having EIIP values between 0.11 and 0.14 Ry and AQVN values>3.3.

The methods of treating a pathological condition additionally based on the flagellin agonist can be practiced in conjunction with other therapies. For example, for treating cancer, the methods of the invention can be practiced prior to, during, or subsequent to conventional cancer treatments such as surgery, chemotherapy, including administration of cytokines and growth factors, radiation or other methods known in the art. Similarly, for treating pathological conditions which include infectious disease, the methods of the invention can be practiced prior to, during, or subsequent to conventional treatments, such as antibiotic administration, against infectious agents or other methods known in the art. The methods of the invention can be administered in conjunction with other methods known in the art and at various times prior, during or subsequent to initiation of conventional treatments.

It is understood that modifications which do not substantially affect the activity of the various embodiments of this invention are also included within the definition of the invention provided herein. Accordingly, the following examples are intended to illustrate but not limit the present invention.

In particular, all the amino acid sequences according to the present invention have been considered effective in treating all the above identified diseases. In fact, the only important issue is that said sequences present a common value ranging from 0.404 to 0.407 as a dominant peak of frequency in the informational spectrum analysis. Changes of amino acids within the sequence of the peptide or polypeptide as herein defined do not alter the efficacy of the functionality since the common value has been found constant.

### Example 1

### Analysis of Amino Acid Sequences by the ISM

In the first step of the ISM analysis each constitutive element (amino acid) in an analyzed sequence is represented by corresponding EIIP value. For calculation of EIIP the following expression derived from the “general model pseudopotentiar” [Veljkovic V. and Slavic I., Phys. Rev. Lett., 21, 105 (1972); Veljkovic V., Phys. Lett., 45A. 41 (1973)] was used:

W=0.25Z*sin(1.04πZ*)/2 Eq. (1)

where Z* is the average quasivalence number (AQVN) determined by

Z*=Σ^{m}nᵢZᵢ/N Eq. (2)

where Z is the number of valence electrons of the i-th atomic component, n is the number of atoms of the i-th component, m and N are the number of atomic components and total number of atoms in the side group, respectively. The values of EIIP for side groups of amino acids calculated in accord with Eq. (1) are given in the following Table 1. The EIIP values calculated according to equations (1) and (2) are expressed in Rydbergs (Ry) units.

TABLE 1
The electron-ion interaction potential (EIIP) used to
encode amino acids

| Amino | |
|---|---|
| acid | EIIP [Ry] |
| Leu | 0.0000 |
| Ile | 0.0000 |
| Asn | 0.0036 |
| Gly | 0.0050 |
| Glu | 0.0057 |
| Val | 0.0058 |
| Pro | 0.0198 |
| His | 0.0242 |
| Lys | 0.0371 |
| Ala | 0.0373 |
| Tyr | 0.0516 |
| Trp | 0.0548 |
| Gln | 0.0761 |
| Met | 0.0823 |
| Ser | 0.0829 |
| Cys | 0.0829 |
| Thr | 0.0941 |
| Phe | 0.0946 |
| Arg | 0.0959 |
| Asp | 0.1263 |

The numerical serie determined in this way is fmite-lenght deterministic discrete signal containing information corresponding to selective long-distance interaction among biological macromolecules. In order to analyze this information, the obtained numerical sequence was subjected to Fast Fourier Transformation (FFT), which is defined as follows [Rabiner R., L. and old B., Theory and applications of digital processing. Prentice-Hall Inc., Englewood 1975)]:

X(n)=Σ×(m)β^{N},n=1,2, . . . N/2,m=0 Eq. (3)

Here x(m) is the m-th of a given numerical series, and X(n) are coefficients of FFT. The coefficients are describing the amplitude, phase, and frequency of sinusoids from which original signal consists. The absolute value of complex FFT coefficients determines the amplitude spectrum which is in the ISM defined as informational spectrum (IS) and represented by the following equation:

S(n)=X(n)X*(n)=|X(n)|²,n=1,2, . . . ,N/2 Eq. (4)

It was assumed that points in analyzed numerical sequences are equidistant with the distance d=1. In this case the maximal frequency in the spectrum is Fₘₐₓ=1/2d=0.5. It is important to note that the frequency range is independent on the number of points in the sequence. The total number of points in the sequence (i.e. number of amino acids in the analyzed primary structure) influences only the resolution of IS. In the case of an N-point sequence, the resolution equals 1/N. The minimal length of sequence that can be analyzed by ISM is determined by the desired resolution of the spectrum. Therefore, this number is determined by the expected number of peaks which are to be strictly separated and cannot be exactly defined. The minimal length of sequence which can be analyzed by ISM with suitable accuracy is 16 amino acids. The IS frequencies correspond to the distribution of structural motifs with defined physicochemical properties determining a biological function of a protein. When comparing proteins, which share the same biological or biochemical function, the ISM technique allows the detection of code/frequency pairs which are specific for their common biological properties, or which correlate with their specific interaction. These common informational characteristics of sequences are determined by cross-spectrum or consensus informational spectrum (CIS). A CIS of N spectra is obtained by the following equation:

C(j)=ΠS(i,j) (5)

where S(i,j) is the j-th element of the i-th power spectrum and C(j) is the j-th element of CIS. Thus, CIS is the Fourier transform of the correlation function for the spectrum. Thus, any spectral component (frequency) not present in all compared informational spectra is eliminated. Peak frequencies in CIS represent the common information encoded in the primary structure of the sequences analyzed. In this way, the information primarily defined by the sequence of symbols representing amino acids is presented in spectral form which is more suitable for mathematical analysis. It is important to note that ISM does not influence this information and represents only a tool for its analysis (like the prism which decomposes the white light in its spectral components). Each frequency in the IS represents a particular informational component encoded in the primary structure by regularly distributed structural motifs with similar electronic properties. In other words, within a protein sequence there exists one or more amino acids motifs which have similar electronic properties. Said properties can be represented by means of frequencies in an IS spectrum. Said motifs are those responsible for the function of the protein. All frequencies calculated by the ISM procedure are determined with accuracy of ±0.004.

The significance of particular frequency is determined by the parameter S/N (signal-to noise ratio) representing ratio between the amplitude value on the particular frequency and the average value of all other amplitudes in IS. The S/N value directly depends on the informational similarity between analyzed sequences and on their number in the cross-spectrum.

### Example 2

### Application of the Technique from Example 1 on Analysis of Flagellin Molecules

The analysis procedure comprises the following steps in succession:
1. each amino acid sequences of the 99 flagellin proteins was converted to the numerical sequence by representing each ammo acid with the corresponding value of the EIIP according to the above Table 1;
2. this numerical sequence was converted into a numerical spectrum using fast Fourier transform (hereinafter FFT);
3. spectra were mutually compared using cross-spectral analysis with the aim to extract common frequency components;
4. a spectrum was obtained using the FFT for a selected flagellin protein at the extracted common frequency F(0.405);
5. two domains of the selected flagellin protein, i.e. two distinct and separate amino acid sequences within the protein sequence, have been identified in said spectrum as parts of flagellin which is essential for its biological property represented by the frequency F(0.405).

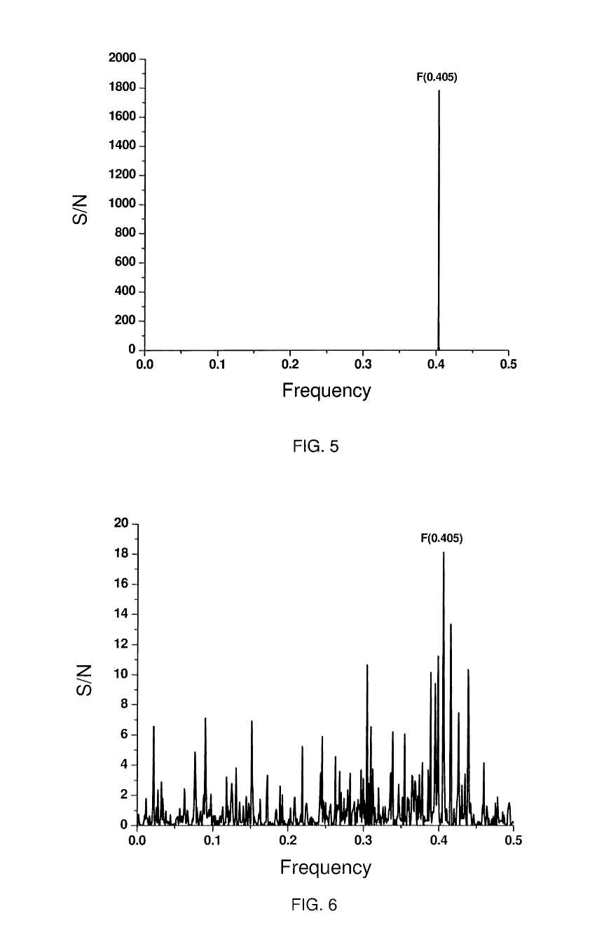

From the ISM analysis of 99 flagellins molecules from various species (FIG. 5) one can deduce the frequency F(0.405) as a common spectral characteristic of analyzed sequences.

All frequency components are determined with accuracy of ±0.004.
From the obtained results, it is possible to conclude that information which is essential for biological activity of the analyzed flagellin molecules is represented with the frequency F(0.405).

### Example 3

### Application of the Technique from Example 1 on Analysis of Flagellin/TLR-5 Interaction

The informational spectrum of flagellin from Salmonella enterica subsp. enterica serovar Edinburgh [Acce. No. ADU54190] calculated by the procedure 24 described in the Example 1 is given in FIG. 4.

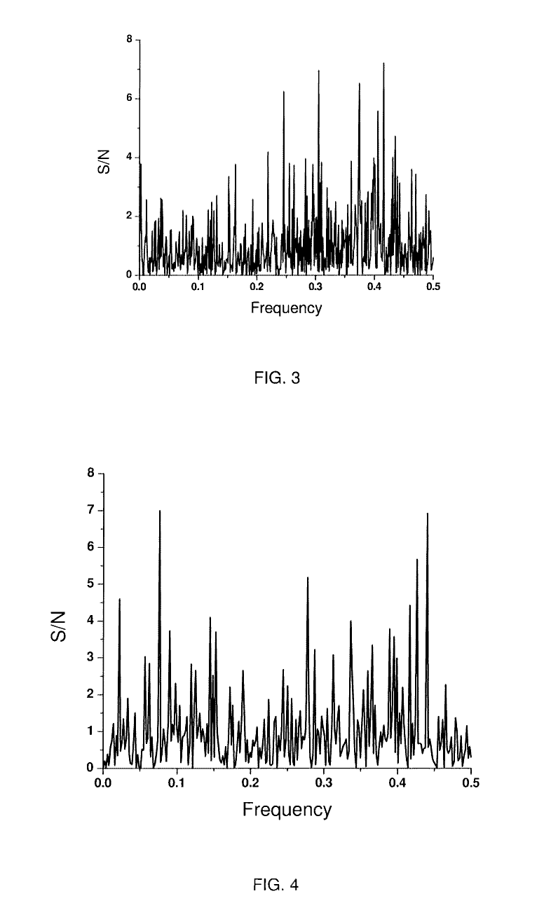

The informational spectrum of TLR-5 calculated by the procedure described in the Example 1 is given in FIG. 3.

The cross-spectrum of flagellin from Salmonella enterica subsp. enterica serovar Edinburgh [Acce. No. ADU54190] and TLR-5 calculated by the procedure described in the Example 1 is given in FIG. 6. From the presented results, it is possible to conclude that information which is essential for interaction between flagellin and TLR-5 is represented with the frequency F(0.405).

### Example 4

### Application of the Technique from Example 1 on Mapping of TLR-5 Interacting Sites on Flagellin

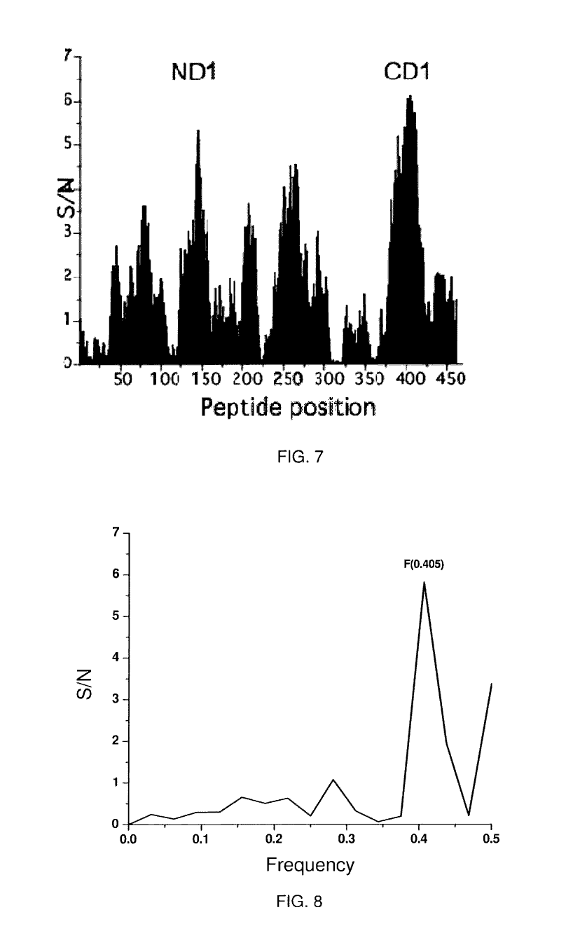

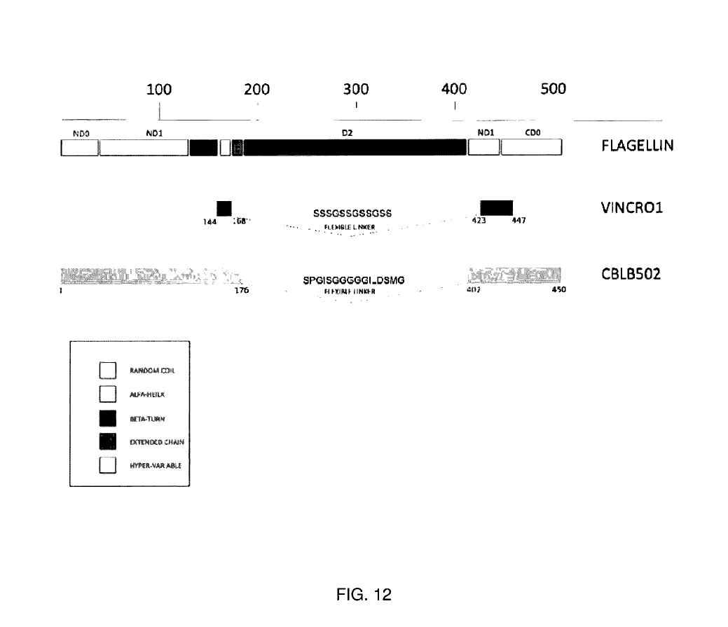

The computer-assisted scanning of flagellin from Salmonella enterica subsp. enterica serovar Edinburgh [Acce. No. ADU54190] for TLR-5 interacting sites is performed by the procedure described in the Example 1. In particular, the analysis has been carried out putting the selected flagellin protein in an electric field and measuring the oscillations at the above prefixed frequency value of 0.405. According to results of this analysis presented in FIG. 7 it can be concluded that essential contribution to the frequency component F(0.405) in informational spectrum of flagellin give domains ND1 (residues 144-168) and CD1 (residues 423-447). In particular, as shown in FIG. 12, from the comparison between the length of the flagellin protein, the CBLB502 and the VINCRO1 polypeptides, it can be observed that VINCRO1 is much more small than both the flagellin and the CBLB502 polypeptide. The comparison between VINCRO1 and the flagellin antagonist CBLB502 schematically shown in FIG. 12 highlights the significative difference both in length 62 residues for VINCRO1, excluding the polyhistidine tag and cloning procedure-derived residues, compared to 236 for CBLB502 excluding the polyhistidine tag and cloning procedure-derived residues, and in composition between the two constructs. Further, the extension of the two ND1 and CD1 domains has been selected considering the single position corresponding to the peak of the S/N value for each domain and counting around said value peak the residues showing more than 90% conservation among flagellins. In this manner, the production of the synthetic polypeptide VINCRO1 is much more easy and reliable.

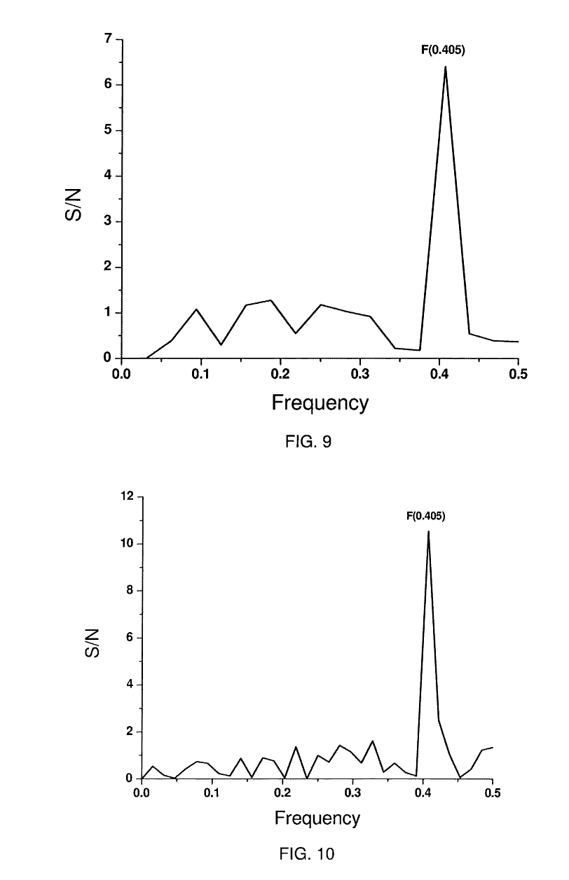

Informational spectra of flagellin domains ND1 and CD1 are given in FIG. 8 and FIG. 9, respectively. It is to be noticed that each plot of the FIGS. 8 and 9 shows a peak at the frequency value F 0.405.

### Example 5

### Design of the Flagellin Mimetic (SEQ ID NO: 2) Using Data Obtained in Example 3

TLR-5-binding peptides ND1 and CD1 derived from flagellin of Salmonella enterica subsp. enterica serovar Edinburgh [Acce. No. ADU54190] are connected with the flexible linker SSSGSSGSSGSS in order to mimic the structure of the native flagellin. The primary structure of this polypeptide (hereafter referred as VINCRO1) is in accord with Claims 1-6 and with the general reference sequence (SEQ ID NO: 1 and SEQ ID NO 3). The Informational spectrum of this flagellin mimetic is given in FIG. 10. As can be seen, the informational spectrum of this protein construct contains only one pick on the frequency F(0.405) which characterizes interaction between flagellin and TLR-5.

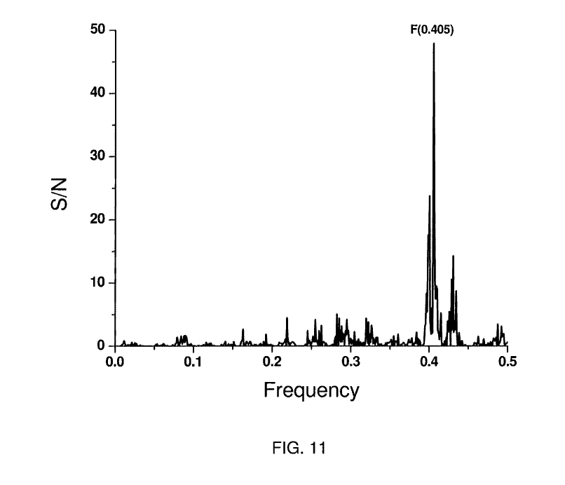

The cross-spectrum between the flagellin mimetic and TLR-5 is given in FIG. 11. Dominant peak in this cross-spectrum on the frequency F(0.405) suggests specific interaction between these proteins.

### Example 6

### Cloning and Expression of the Flagellin Mimetic Described in Example 5

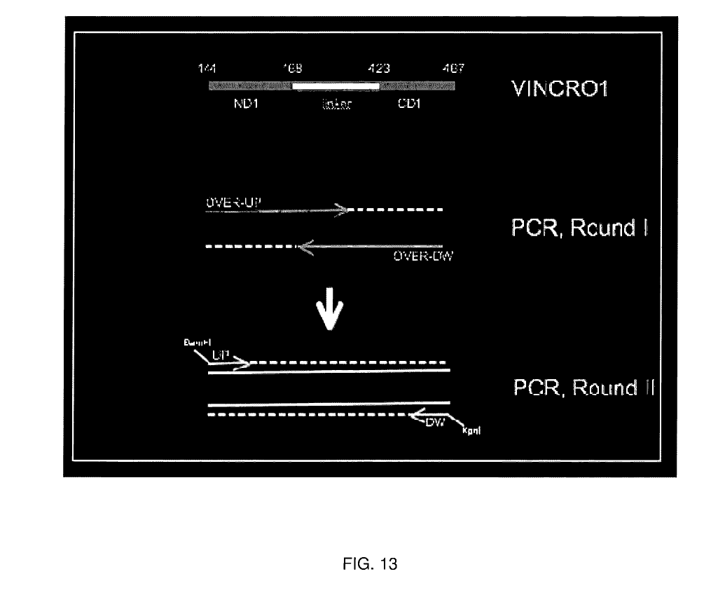

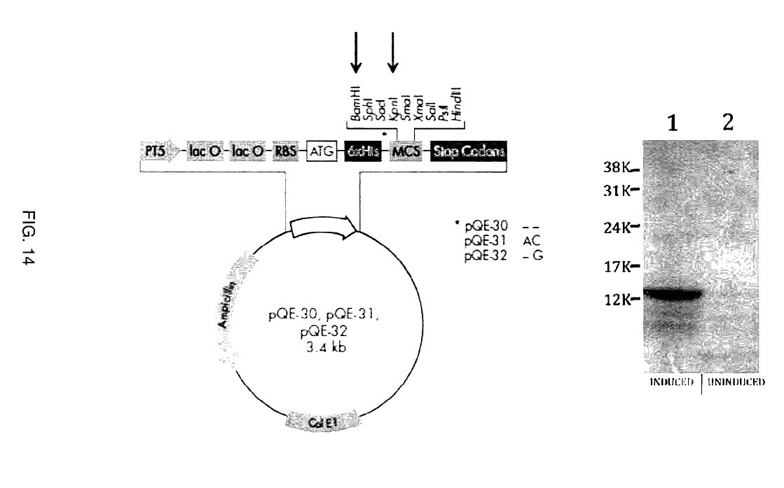

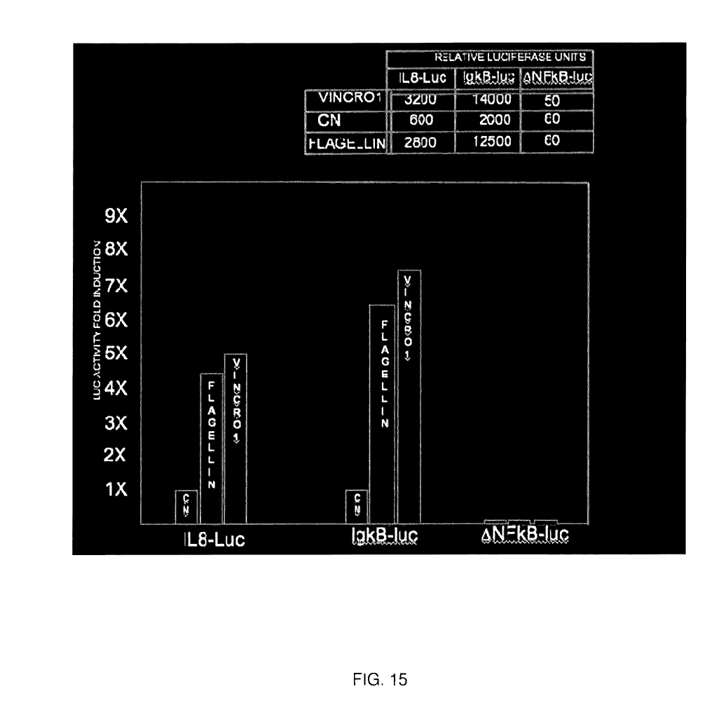

Regions 144-168 and 423-447 of N- and C-terminal D1 domains of flagellin, respectively, were fused to a flexible linker using a PCR-based strategy (FIG. 13): in a first PCR round, partially overlapping primers covering the 144-168 ND1 flagellin sequence plus the first 10 residues of the flexible linker (over-up: 5′-ctgaccatccaggttggtgccaacgacggtgaaactatc gatatcgatctgaagcagatcaactctcagaccctgtcttcctctggctcttccggctcttcc-3′) and the last 10 residues of the flexible linker plus 423-447 CD1 flagellin sequence (over-down 5′-agcggagttaaaacggttctgtaccgcacccaggtcagaacgcagcgcatccacctgcgccagcgcggcatcaat ggaagagcccgaagagccggaagagccaga-3′) were subjected to extension for 26 cycles. The agarose-gel purified product was amplified in a second PCR round for 35 cycles by using external primers with appended BamHI and KpnI adaptor sequences (up: 5′-ggggatccctgaccatccaggttggtgcc 3′; and down: 5′ ggggttaccagcggagttaaaacggttc-3′). The resulting synthetic DNA was cloned in the pQE-30 plasmid (Promega), that transformed in MI5 bacterial strain allowed the expression of the cloned sequence fused at the N-terminus to an 5′ histidine tag following IPTG induction. The bacterial pellet was subjected to two thermal shock cycles followed by sonication and the released VINCRO1 was purified under native conditions from bacteria by means of Ni-NTA Agarose (Promega). FIG. 14 shows the vector scheme, (left), with arrows indicating the cloning sites, an SDS-PAGE of the purification product (right) obtained from an IPTG-induced culture (lane1), and the purification product of an uninduced culture (lane2), used as a negative control in the following experiments. Amino acid sequence of VINCRO1 consists in a cloning-derived residues plus polyhistidine tag, the Flagellin derived binding polypeptide ND1, a synthetic flexible linker and the Flagellin derived binding polypeptide CD1:

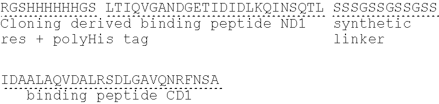

### Example 7

### Evaluation of the NF-kB Activation Induced by VINCRO1 Peptide Obtained in Example 6

### Transient Transfectios and Luciferase Assay

To evaluate the activity, VINCRO1 peptide was tested in a well established NF-kB reporter system assay in comparison with commercially available ultrapure flagellin (Invitrogen, Life Technologies, Carlsbad, Calif., cat. tirl-pstfla) as positive control and an unrelated recombinant protein as negative control (CN═C1q domain of EMILIN1). All transfections were performed in triplicate in 24-well plates. Approximately 2×10⁵ CACO-2 cells per well were seeded 24 h before transfection. Following the manufacturer's instructions, plasmids were transfected into cells using FUGENE transfection reagent (Promega, Medison, Wis.). Briefly, 0.8 μg of reporter plasmids carrying different NF-kB responsive elements (IL8-Luc, IGkB-Luc) plus 0.05 μg of pCMV-LacZ vector were diluted with Opti-MEM and then mixed with diluted liposomes. After 20 min of incubation at room temperature, the mixtures were added to each well. At 24 h post-transfection, cells were challenged with 10 microg/ml of VINCRO1, or molar equivalent of flagellin as positive control and the C1q of EMILIN1 as negative control. Luciferase assays were performed using the luciferase assay system (Promega); β-Galactosidase activity was used as an internal control. Each experiment was conducted a minimum of three times. As shown in FIG. 15, VINCRO1 exhibits an activity at least comparable with that of the full-length flagellin. To confirm the VINCRO1 specificity, lysates of cell transfected (as above) with a mock vector (delta NFkB-Luc) showed no luciferase activity.

### Example 8

### Evaluation of the In Vivo Protective Role of VINCRO1 Peptide

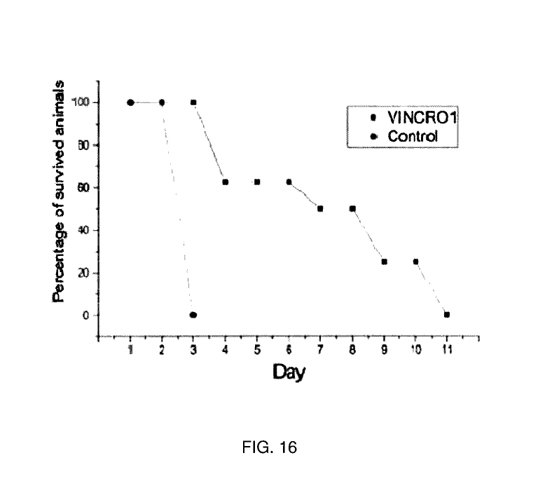

To evaluate the protective role of the peptide in adult Wistar rats 6 week old males, eight animals per group were injected subcutaneously (s.c.) with 0.1 mg/kg of VINCRO1 solution in PBS. One hour later, rats were irradiated for about 30 min with a single total dose of 9.6 Gy and survival was monitored daily (FIG. 16).

1. An isolated polypeptide which polypeptide presents substantially the same frequencies as natural flagellin in the informational cross-spectrum with human toll-like receptor-5 (TLR-5), obtained by Fourier transformation according to the method of the informational spectrum analysis, wherein in the informational spectrum as a dominant the frequency component F is in the region 0.404-0.407, preferably 0.405.

2. An isolated polypeptide according to claim 1 having the general formula R1-L-R2 wherein: R1 is an amino terminal sequence, L is flexible peptide linker comprising 10-14 amino acids, R2 is the carboxy terminal sequence, wherein both R1 and R2 have in their informational spectrum the dominant frequency component belonging to the frequency interval 0.404-0.407.

3. An isolated polypeptide according to claim 2, wherein R1 and R2 sequences consist in the general formula X1-X2-X3-X4-X5-X6-X7-X8-X9-X10-X11-X12-X13-X14-X15-X16-17-X18-X19-X20-X21-X22-X23-X24-X25 (SEQ ID NO 3), wherein X1 is Val, Leu, lie or Asn, X2 is Thr or Arg, X3 is Val, Leu, Ile or Asn, X4 is Met or Gln, X5 is Val, Leu, lie or Asn, X6 is Val, Leu, lie or Asn, X7 is Val, Leu, lie, Asn or Ala, X8 is Val, Leu, Ile or Asn, X9 is Asp, X10 is Val, Leu, Ile or Asn, X11 is Gly or Glu, X12 is Thr or Arg, X13 is Val, Leu, lie or Asn, X14 is Asp, X15 is Val, Leu, Ile or Asn, X16 is Asp, X17 is Val, Leu, Ile or Asn, X18 is Ala, X19 is Gln or Met, X20 is Val, Leu, Ile or Asn, X21 is Val, Leu, lie or Asn, X22 is Met or Gln, X23 is Met or Gln, X24 is Thr or Arg and X25 is Val, Leu, lie or Asn.

4. An isolated polypeptide according to claim 3, wherein R1 and R2 sequences are selected from the group consisting in:
(SEQ ID NO 4)
IRIMIIVIDIERIDIDVAMIVMQRI,
(SEQ ID NO 5)
IRIMIIVIDIGRIDIDVAMIVMQRI,
(SEQ ID NO 6)
IRIMINVIDIERIDIDVAMIVMQRI,
(SEQ ID NO 7)
IRIMIVVIDIERIDIDVAMIVMQRI,
(SEQ ID NO 8)
IRIMINVIDIGRIDIDVAMIVMQRI,
(SEQ D NO 9)
IRIMIVVIDIGRIDIDVAMIVMQRI,
(SEQ ID NO 10)
IRIMIIVIDIERIDIDVAMIVQQRI,
(SEQ ID NO 11)
IRIMIIVIDIERIDIDNAMIVMQRI,
(SEQ ID NO 12)
IRIMIIVIDIGRIDIDVAMIVQQRI,
(SEQ ID NO 13)
IRIMINVIDIERIDIDVAMIVQQRI,
(SEQ ID NO 14)
IRIMIVVIDIERIDIDVAMIVQQRI,
(SEQ ID NO 15)
IRIMIIVIDIGRIDIDNAMIVMQRI,
(SEQ ID NO 16)
IRIMINVIDIERIDIDNAMIVMQRI,
(SEQ ID NO 17)
IRIMINVIDIGRIDIDVAMIVQQRI,
(SEQ ID NO 18)
IRIMIVVIDIERIDIDNAMIVMQRI,
(SEQ ID NO 19)
IRIMIVVIDIGRIDIDVAMIVQQRI,
(SEQ ID NO 20)
IRIMIIVIDIETIDIDVAMIVMQRI,
(SEQ ID NO 21)
LTIQVGANDGETIDIDLKQINSQTL,
(SEQ ID NO 22)
IRIMIVVIDIGRIDIDNAMIVMQRI,
(SEQ ID NO 23)
IDIMIIVIDIGTIDIDVAMIVMQRI,
(SEQ ID NO 24)
NRIMIIVIDIGRIDIDVAMIVMQRI,
(SEQ ID NO 25)
IDIMINVIDIETIDIDVAMINMQRI,
(SEQ ID NO 26)
NRIMINVIDIERIDIDVAMIVMQRI,
(SEQ ID NO 27)
IRIMIIVIDIERIDIDNAMIVQQRI,
(SEQ ID NO 28)
IDAALAQVDALRSDLGAVQNRFNSA,
(SEQ ID NO 29)
IRIMIIVIDIERIDIDVAMINMQRI,
(SEQ ID NO 30)
NRIMIVVIDIERIDIDVAMIVMQRI,
(SEQ ID NO 31)
IRIMIVVIDIETIDIDVAMIVMQRI,
(SEQ ID NO 32)
IRIMINVIDIGTIDIDVAMIVMQRI,
(SEQ ID NO 33)
NRIMINVIDIGRIDIDVAMIVMQRI,
(SEQ ID NO 34)
IRIMINVIDIERIDIDNAIMIVQRI,
(SEQ ID NO 35)
IRIMIIVIDIGRIDIDNAMIVQQRI,
(SEQ ID NO 36)
IRIMIIVIDIGRIDIDVAMINMQRI,
(SEQ ID NO 37)
NRIMIVVIDIGRIDIDVAMIVMQRI,
(SEQ ID NO 38)
IRIMIVVIDIGTIDIDVAMIVMQRI,
(SEQ ID NO 39)
IRIMIVVIDIERIDIDNAMIVQQRI,
(SEQ ID NO 40)
IRIMINVIDIERIDIDVAMINMQRI,
(SEQ ID NO 41)
IRIMINVIDIGRIDIDNAMIVOQRI,
(SEQ ID NO 42)
NRIMIIVIDIERIDIDVAMIVQQRI,
(SEQ ID NO 43)
IRIMIINIDIERIDIDVAMIVMQRI,
(SEQ ID NO 44)
IRIMIIVIDIETIDIDVAMIVQQRI,
(SEQ ID NO 45)
IRIMIVVIDIERIDIDVAMINMQRI,
(SEQ ID NO 46)
VRIMIIVIDIERIDIDVAMIVMQRI,
(SEQ ID NO 47)
LRIMIVVIDIGRIDIDNAMIVQQRI,
(SEQ ID NO 48)
LRIMINVIDIGRIDIDVAMINMQRI,
(SEQ ID NO 49)
LRIMIIVIDIETIDIDNAMIVMQRI,
(SEQ ID NO 50)
ITIMIIVIDIERIDIDVAMIVMQRI,
(SEQ ID NO 51)
NRIMINVIDIERIDIDVAMIVQQRI,
(SEQ ID NO 52)
NRIMIIVIDIERIDIDNAMIVMQRI,
(SEQ ID NO 53)
IRIMIINIDIGRIDIDVAMIVMQRI.

5. An isolated polypeptide according to claim 2, 3 or 4, wherein the peptide linker L consists in the amino acid sequence SSSGSSGSSGSS.

6. An isolated polypeptide according to any one of claims 1-5 consisting in the amino acid sequence
LTIQVGANDGETIDIDLKQINSQTLSSSGSSGSSGSSIDAALAQVDALRS
DLGAVQNRFNSA

7. A synthetic polynucleotide coding for an isolated polypeptide according to any preceding claim.

8. An expression vector comprising a polynucleotide according to claim 7.

9. A pharmaceutical composition comprising a polypeptide according to any of claims 1 to 6 in admixture with one or more pharmaceutically acceptable carriers or excipients.

10. A pharmaceutical composition according to claim 9 in combination with a radioprotectant, antiviral or antibiotic substances.

11. The composition of claim 10, wherein the radioprotectant is an antioxidant.

12. The composition of claim 11, wherein the antioxidant is selected from the group of molecules having the EIIP value between 0 and 0.10 Ry and the AQVN value<2.4.

13. The composition of claim 11, wherein the antioxidant is selected from the group of compounds having EIIP values between 0.11 and 0.14 Ry and the AQVN values>3.3.

14. An antibody capable of binding a peptide as defined in claims 1-6.

15. A peptide conjugated with the antibody of claim 14, for use as an agonist or antagonist of TLR-5 receptor.

16. A vaccine adjuvant comprising, as an active ingredient a polypeptide according to any one of claims 1 to 6.

17. A method of treating apoptosis-mediated tissue damage caused by radiation in a mammal comprising administering to a mammal in need thereof a composition comprising a polypeptide according to any one of claims 1 to 6, a composition according to claims 9-13 or an antibody according to claim 14, wherein the apoptosis is attributable to a cellular stress.

18. The method of claim 17, wherein the mammal is a human.

19. The method of claim 17, wherein the radiation is cancer radiotherapy.

20. The method of claim 17, wherein the composition is administered prior to, together with, or after radiation.

21. Methods for detecting normal or abnormal expression of TLR-5 polypeptide in an isolated sample or in vivo diagnostic imaging procedures using a polypeptide according to the claims 1-6, a composition according to claims 9-13 and antibodies according to claim 14.
